(19) United States
(12) Patent Application Publication
(10) Pub. No.: US 2005/0064005 A1
(43) Pub. Date: Mar. 24, 2005

(54) Active agent delivery systems including a miscible polymer blend, medical devices, and methods

(76) Inventors: Thomas Q. Dinh, Minnetonka, MN (US); Randall V. Sparer, Andover, MN (US); SuPing Lyu, Maple Grove, MN (US); Kiem Dang, Blaine, MN (US); Christopher M. Hobot, Tonka Bay, MN (US)

Correspondence Address: MUETING, RAASCH & GEBHARDT, P.A., P.O. BOX 581415, MINNEAPOLIS, MN, 55458, US

(21) Appl. No.: 10/916,162

(22) Filed: Aug. 11, 2004

Related U.S. Application Data
(60) Provisional application No. 60/494,979, filed on Aug. 13, 2003.

(51) Int. Cl. A61K 9/14

(52) U.S. Cl. 424/422; 424/486

(57) ABSTRACT

An active agent delivery system that includes two or more active agents and two or more layers of polymers; wherein at least one layer includes miscible polymer blend comprising two or more miscible polymers; and further wherein delivery of at least one active agent occurs predominantly under permeation control.

### CROSS-REFERENCE TO RELATED APPLICATIONS

The present application claims the benefit of U.S. Provisional Application No. 60/494,979, filed on 13 Aug. 2003, which is incorporated herein by reference in its entirety.

### BACKGROUND

A polymeric coating on a medical device may serve as a repository for delivery of an active agent (e.g., a therapeutic agent) to a subject. For many such applications, polymeric coatings must be as thin as possible. Polymeric materials for use in delivering an active agent may also be in various three-dimensional shapes.

Conventional active agent delivery systems suffer from limitations that include structural failure due to cracking and delamination from the device surface. Furthermore, they tend to be limited in terms of the range of active agents that can be used, the range of amounts of active agents that can be included within a delivery system, and the range of the rates at which the included active agents are delivered therefrom. This is frequently because many conventional systems include a single polymer.

Thus, there is a continuing need for active agent delivery systems with greater versatility and tunability, particularly when more than one active agent is used.

### SUMMARY OF THE INVENTION

The active agent delivery systems of the present invention typically include two or more active agents and two or more layers of polymers (preferably, up to 20 layers and more preferably up to hundreds or even thousands of layers); wherein at least one layer includes a miscible polymer blend that includes two or more miscible polymers. The system is designed such that the delivery of at least one of the active agents occurs predominantly under permeation control. In this context, “predominantly” with respect to permeation control means that at least 50%, preferably at least 75%, and more preferably at least 90%, of the total active agent load is delivered by permeation control.

Permeation control is typically important in delivering an active agent from systems in which the active agent passes through a miscible polymer blend having a “critical” dimension on a micron-scale level (i.e., the diffusion net path is no greater than about 1000 micrometers, although for shaped objects it can be up to about 10,000 microns). For a multilayer system, the critical dimension is the dimension of a blend layer or layers that play a role in the controlled permeation of the active agent(s). Furthermore, it is generally desirable to select polymers for a particular active agent that provide desirable mechanical properties without being detrimentally affected by nonuniform incorporation of the active agent.

The present invention provides active agent delivery systems that have generally good versatility and tunability in controlling the delivery of active agents. Typically, such advantages result from the use of a blend of two or more miscible polymers. These delivery systems can be incorporated into medical devices, e.g., stents, stent grafts, anastomotic connectors, if desired.

A wide variety of constructions can be used in an active agent delivery system that includes two or more active agents and two or more layers of polymers, wherein at least one layer includes a miscible polymer blend that includes two or more miscible polymers. The systems of the present invention can include a wide range of layers (e.g., tens, hundreds, or even thousands). Particularly preferred systems include 2, 3, or 4 layers.

For two-layered systems, the inner layer can include all the active agents and the outer layer can function as a barrier layer. Alternatively, both layers can include one or more active agents.

For three-layered systems, the inner two layers can include all the active agents and the outer layer can function as a barrier layer. Alternatively, the innermost and outermost layers can include all the active agents and the middle layer can function as a barrier layer. Alternatively, all three layers can include one or more active agents.

For four-layered systems, the inner three layers can include all the active agents and the outer layer can function as a barrier layer. Alternatively, one of the two middle layers can function as a barrier layer. Alternatively, two layers can function as barrier layers. Alternatively, all four layers can include one or more active agents.

In one embodiment, at least one active agent is incorporated within the at least one miscible polymer blend layer. Alternatively, the miscible polymer blend layer can initially provide a barrier for the active agent. That is, initially, it does not contain any active agent. The miscible polymer blend layer with at least one active agent incorporated therein can be an inner layer (e.g., the innermost layer)

Various layers of the active agent delivery systems of the present invention can include a single polymer layer. This can form the outermost layer, for example, and when no active agent is present in this layer, it forms a barrier layer. Alternatively, the single polymer layer can include an active agent in which the system further includes a barrier layer overlying the single polymer layer.

Other embodiments can include at least two layers, each of which has at least one active agent incorporated therein. Each layer can include a blend of two or more miscible polymers.

Certain embodiments can include layers of immiscible mixtures of polymers. For example, in one embodiment of at least two layers, an inner layer can include an immiscible mixture of two or more polymers with at least one active agent incorporated therein, and the system can further include a barrier layer overlying the immiscible polymer mixture layer, wherein the barrier layer does not include an active agent initially, and further wherein the barrier layer includes a miscible polymer blend.

Certain embodiments can include a concentration gradient of at least one of the active agents such that the concentration of at least one active agent varies throughout the layers.

Certain embodiments can include the same polymers in each layer in varying amounts such that a concentration gradient is formed.

For certain embodiments, the difference between the solubility parameter of the active agent that is to be released faster and to be present in a greater amount (i.e., greater load) and the volume average solubility parameter of the blend of the two or more miscible polymers is smaller than the differences between the solubility parameter of each of the other one or more active agents and the volume average solubility parameter of the blend of the two or more miscible polymers.

The present invention also provides medical devices (e.g., stents, stent grafts, anastomotic connectors) that include such active agent delivery systems. Such medical devices include, for example, a substrate surface, a polymeric undercoat layer adhered to the substrate surface, and an active agent delivery system adhered to the polymeric undercoat layer.

The present invention also provides methods for delivering two or more active agents to a subject. In one embodiment, a method of delivery includes: providing an active agent delivery system as described above and contacting the active agent delivery system with a bodily fluid, organ, or tissue of a subject.

Herein, “predominantly” in the context of permeation control means that at least 50% (preferably at least 75%, and more preferably at least 90%) of the total load of at least one active agent is delivered by permeation control. Preferably, all the active agents are delivered under permeation control.

The term “permeability” is the diffusivity times solubility.

The term “molar average solubility parameter” means the average of the solubility parameters of the blend components that are miscible with each other and that form the continuous portion of the miscible polymer blend. These are weighted by their molar percentage in the blend, without the active agent incorporated into the polymer blend.

The term barrier layer refers to a polymer layer that controls the rate of release of the active agent(s). It does not prevent permeation; rather, it slows the rate of permeation and/or increases the lag time. It typically is a discrete layer and prevents the smearing of active agents.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE DRAWINGS

The invention will be further explained with reference to the drawings. FIGS. 1 and 6 are idealized, not to scale, and intended to be merely illustrative and non-limiting.

FIG. 1 is a cross-section of a stent coated with a three-layer active agent delivery system containing mycophenolic acid and sulfasalazine with a primer layer.

FIG. 2 is a graph of the release kinetics of mycophenolic acid and sulfasalazine from the active agent delivery system shown in FIG. 1 without a barrier layer.

FIG. 3 is a graph of the release kinetics of mycophenolic acid and sulfasalazine from the active agent delivery system shown in FIG. 1.

FIG. 4 is a graph of the release kinetics of sulfasalazine from various blends of TECOPLAST/TECOPHILIC polyurethanes.

FIG. 5 is a graph of the release kinetics of mycophenolic acid and sulfasalazine from an alternative active agent delivery system of the present invention.

FIG. 6 is a cross-section of a stent coated with a two-layer active agent delivery system containing podophyllotoxin and sulfasalazine with a primer layer.

FIG. 7 is a graph of the release kinetics of podophyllotoxin and sulfasalazine from the active agent delivery system shown in FIG. 6.

FIG. 8 is a cross-section of a stent coated with a two-layer active agent delivery system containing etoposide (EP) and sulfasalazine with a primer layer.

FIG. 9 is a graph of the release kinetics of etoposide and sulfasalazine from the active agent delivery system shown in FIG. 8.

### DETAILED DESCRIPTION OF ILLUSTRATIVE EMBODIMENTS

The present invention provides active agent delivery systems that include two or more active agents for delivery to a subject and two or more layers of polymers, wherein at least one layer includes a miscible polymer blend that includes two or more miscible polymers. The delivery systems can include a variety of polymers as long as at least two of them are miscible as defined herein.

A miscible polymer blend can be used in combination with two or more active agents in the delivery systems of the present invention in a variety of formats as long as the miscible polymer blend controls the delivery of the active agent. That is, a wide variety of constructions can be used in an active agent delivery system that includes two or more active agents and two or more layers of polymers, wherein at least one layer includes a miscible polymer blend that includes two or more miscible polymers. Preferably, at least one active agent dissolutes through at least one polymer blend layer.

The systems of the present invention can include a wide range of number of layers. Particularly preferred systems include at least 2, 3, or 4 layers, although at least 5, 10, 20, 50, 100, and 1000 or more layers can be possible. Although not always easy to discern discrete layers by the eye, each layer is defined by a distinct formulation from the layers adjacent thereto, characterized, for example, by the presence or absence of active agents, different polymers, different active agents, different ratios of the same polymers, different concentration of active agents, etc.

Herein, the “active agent delivery system” excludes any primer layer that may be used to enhance adhesion of the multi-layered system to a surface, such as the surface of a stent, for example. Thus, when referring to an “innermost” layer, this refers to the innermost layer of the active agent delivery system. This does not exclude the use of a primer layer.

For two-layered systems, the inner layer can include all the active agents and the outer layer can function as a barrier layer. Alternatively, both layers can include one or more active agents.

For three-layered systems, the inner two layers can include all the active agents and the outer layer can function as a barrier layer. Alternatively, the innermost and outermost layers can include all the active agents and the middle layer can function as a barrier layer. Alternatively, all three layers can include one or more active agents.

For four-layered systems, the inner three layers can include all the active agents and the outermost layer can function as a barrier layer. Alternatively, one of the two middle layers can function as a barrier layer. Alternatively, two layers can function as barrier layers. Alternatively, all four layers can include one or more active agents.

In one embodiment, at least one active agent is incorporated within the at least one miscible polymer blend layer. Alternatively, the miscible polymer blend layer can initially provide a barrier for the active agent. That is, initially, it does not contain any active agent. Preferably, the miscible polymer blend layer with at least one active agent incorporated therein is an inner layer (e.g., the innermost layer).

A barrier layer, i.e., a discrete layer of one or more polymers that is a rate-limiting layer, can be incorporated into a variety of locations within an active agent delivery system of the present invention. Preferably, at least one layer, and more preferably, at least two layers, of the systems of the present invention are barrier layers (e.g., layers that do not initially include an active agent therein). It can be an inner layer (although not the innermost layer), or it can be an outer layer (e.g., outermost layer), preferably it is the outermost layer. When used in an intermediate layer within a system having three or more layers and between layers containing active agents, the barrier layer also prevents smearing of the active agents. Initially, the barrier layers do not include active agents, but as the agents permeate out of the system, the barrier layer(s) will include one or more active agents.

Alternatively, all outer layers of delivery systems of the present invention can function as barriers for the active agents in the inner layers whether or not they include active agents. For example, in a four-layered system the outer three layers can function as barriers for the active agents in the innermost layer even if each of the outer layers contains at least one or more of the active agents. Similarly, the two outermost layers can function as barriers for active agents in the two innermost layers, and so on.

Various layers of the active agent delivery systems of the present invention can include a single polymer layer. This can form the outermost layer, for example, and when no active agent is present in this layer, it forms a barrier layer. Alternatively, the single polymer layer can include an active agent in which the system further includes a barrier layer overlying the single polymer layer.

In one preferred embodiment, the miscible polymer blend layer includes one or more active agents, and the system further includes a barrier layer overlying the miscible polymer blend layer, wherein the barrier layer does not include an active agent initially, and further wherein the barrier layer includes a single polymer or a miscible polymer blend. This system can also include a layer overlying the barrier layer, wherein the overlying layer includes at least one polymer and at least one active agent incorporated therein. Additionally, this system can further include an outermost layer that does not initially include an active agent therein, i.e., a barrier layer.

Other embodiments can include at least two layers (preferably, at least three layers), each of which has at least one active agent incorporated therein (typically, except the outermost layer). Each layer can include a blend or two or more miscible polymers, and preferably each of these includes at least one active agent.

In one preferred embodiment, a system of the present invention includes at least two layers, wherein an inner layer (preferably, the innermost layer) includes at least one polymer (preferably, it is a miscible polymer blend) with at least one active agent incorporated therein, and the system further includes a barrier layer overlying the inner polymer layer, wherein the barrier layer does not include an active agent initially. The barrier layer can include a miscible polymer blend. This barrier layer can be the outermost layer if desired. Preferably, this system includes at least two inner layers each of which includes at least one polymer with at least one active agent incorporated therein. Additionally, this system can include an outermost layer that includes at least one polymer and at least one active agent incorporated therein.

In one preferred embodiment, a system of the present invention includes at least two layers, wherein an inner layer includes a single polymer with an active agent incorporated therein, and the system further includes a barrier layer overlying the single polymer layer, wherein the barrier layer comprises the miscible polymer blend.

Certain embodiments can include layers of immiscible mixtures of polymers. For example, in one embodiment of at least two layers, an inner layer can include an immiscible mixture of two or more polymers with at least one active agent incorporated therein, and the system can further include a barrier layer overlying the immiscible polymer mixture layer, wherein the barrier layer does not include an active agent initially, and further wherein the barrier layer includes a miscible polymer blend. Preferably, the inner layer that includes an immiscible mixture of two or more polymers with at least one active agent incorporated therein is the innermost layer. Additionally, the system can include a layer overlying the barrier layer, wherein the overlying layer includes at least one polymer and at least one active agent incorporated therein.

In another exemplary embodiment of at least two layers, an inner layer includes an immiscible mixture of two or more polymers with at least one active agent incorporated therein, and the system further includes an outermost barrier layer, wherein the barrier layer does not include an active agent initially, and further wherein the barrier layer includes a miscible polymer blend. Preferably, the inner layer that includes an immiscible mixture of two or more polymers and at least one active agent incorporated therein is the innermost layer.

Certain embodiments can include single polymer layers, with or without active agents incorporated therein. For example, in a preferred embodiment, a system of the present invention includes at least two layers, wherein an inner layer includes a single polymer with at least one active agent incorporated therein, and the system further includes an outermost barrier layer, wherein the barrier layer does not include an active agent initially, and further wherein the barrier layer includes a miscible polymer blend. Preferably, the inner layer that includes a single polymer and at least one active agent incorporated therein is the innermost layer.

Certain embodiments can include a concentration gradient of at least one of the active agents such that the concentration of at least one active agent varies throughout the layers.

Certain embodiments can include the same polymers in each layer in varying amounts such that a concentration gradient is formed.

The active agents are incorporated within the miscible polymer blend such that at least one is delivered from the blend predominantly under permeation control. Preferably, all are delivered predominantly under permeation control.

In the active agent delivery systems of the present invention, at least one active agent is dissolutable through at least one miscible polymer blend layer. That is, at least one active agent can be incorporated into at least one miscible polymer blend layer or it can be in a layer underlying a miscible polymer blend layer such that it must pass through the miscible polymer blend layer. Dissolution is controlled by permeation of the active agent through the miscible polymer blend. That is, the active agent initially dissolves into the miscible polymer blend and then diffuses through the miscible polymer blend under permeation control. In this context, “predominantly” means that at least 50%, preferably at least 75%, and more preferably at least 90% of the total load of at least one active agent (preferably, of all the active agents) is delivered by permeation control.

When at least one active agent is dissoluted under permeation control, at least some solubility of the active agent in the miscible polymer blend is required. Dispersions are acceptable as long as little or no porosity channeling occurs in at least one miscible polymer blend layer during dissolution of at least one active agent and the size of the dispersed domains is much smaller than the critical dimension of a blend layer or layers, and the physical properties are generally uniform throughout the composition for desirable mechanical performance.

If the active agents exceed the solubility of the miscible polymer blend and the amount of insoluble active agent exceeds the percolation limit, then the active agent could be dissoluted predominantly through a porosity mechanism. In addition, if the largest dimension of the active agent insoluble phase (e.g., particles or aggregates of particles) is on the same order as the critical dimension of a miscible polymer blend layer or layers, then the active agent could be dissoluted predominantly through a porosity mechanism.

Because the active agent delivery systems of the present invention preferably have a critical dimension on the micron-scale level, it can be difficult to include a sufficient amount of active agent and avoid delivery by a porosity mechanism.

One can determine if there is a permeation-controlled release mechanism by examining a dissolution profile of the amount of active agent released versus time (t). For permeation-controlled release from an active agent in an outermost layer, the profile is directly proportional to t^{1/2}. For permeation-controlled release from an inner layer, the profile is directly proportional to t.

Miscible polymer blends are advantageous because they can provide greater versatility and tunability for a greater range of active agents than can conventional systems that include immiscible mixtures or only a single polymer, for example. That is, using two or more polymers, at least two of which are miscible, can generally provide a more versatile active agent delivery system than a delivery system with only one of the polymers. A greater range of types of active agents can typically be used. A greater range of amounts of an active agent can typically be incorporated into and delivered from (preferably, predominantly under permeation control) the delivery systems of the present invention. A greater range of delivery rates for an active agent can typically be provided by the delivery systems of the present invention. At least in part, this is because of the use of a miscible polymer blend that includes at least two miscible polymers. It should be understood that, although the description herein refers to two polymers, the invention encompasses systems that include more than two polymers, as long as a miscible polymer blend is formed that includes at least two miscible polymers.

A miscible polymer blend of the present invention has a sufficient amount of at least two miscible polymers to form a continuous portion, which helps tune the rate of release of the active agent. Such a continuous portion (i.e., continuous phase) can be identified microscopically or by selective solvent etching. Preferably, the at least two miscible polymers form at least 50 percent by volume of a miscible polymer blend. Each of at least two polymers is present in an amount of 0.1 wt-% to 99.9 wt-%, based on the total weight of the polymers.

A miscible polymer blend can also optionally include a dispersed (i.e., discontinuous) immiscible portion. If both continuous and dispersed portions are present, the active agent can be incorporated within either portion. Preferably, the active agent is loaded into the continuous portion to provide delivery of the active agent predominantly under permeation control. To load the active agent, the solubility parameters of the active agent and the portion of the miscible polymer blend a majority of the active agent is loaded into are matched (typically to within no greater than about 10 J^{1/2}/cm^{3/2}, preferably, no greater than about 5 J^{1/2}/cm^{3/2}, and more preferably, no greater than about 3 J^{1/2}/cm^{3/2}). The continuous phase controls the release of the active agent regardless of where the active agent is loaded.

A miscible polymer blend, as used herein, encompasses a number of completely miscible blends of two or more polymers as well as partially miscible blends of two or more polymers. A completely miscible polymer blend will ideally have a single glass transition temperature (Tg), preferably one in each phase (typically a hard phase and a soft phase) for segmented polymers, due to mixing at the molecular level over the entire concentration range. Partially miscible polymer blends may have multiple Tg's, which can be in one or both of the hard phase and the soft phase for segmented polymers, because mixing at the molecular level is limited to only parts of the entire concentration range. These partially miscible blends are included within the scope of the term “miscible polymer blend” as long as the absolute value of the difference in at least one Tg (Tg_{polymer 1}-Tg_{polymer 2}) for each of at least two polymers within the blend is reduced by the act of blending. Tg's can be determined by measuring the mechanical properties, thermal properties, electric properties, etc. as a function of temperature.

A miscible polymer blend can also be determined based on its optical properties. A completely miscible blend forms a stable and homogeneous domain that is transparent, whereas an immiscible blend forms a heterogeneous domain that scatters light and visually appears turbid unless the components have identical refractive indices. Additionally, a phase-separated structure of immiscible blends can be directly observed with microscopy. A simple method used in the present invention to check the miscibility involves mixing the polymers and forming a thin film of about 10 micrometers to about 50 micrometers thick. If such a film is generally as clear and transparent as the least clear and transparent film of the same thickness of the individual polymers prior to blending, then the polymers are completely miscible.

Miscibility between polymers depends on the interactions between them and their molecular structures and molecular weights. The interaction between polymers can be characterized by the so-called Flory-Huggins parameter (χ). When χ is close to zero (0) or even is negative, the polymers are very likely miscible. Theoretically, χ can be estimated from the solubility parameters of the polymers, i.e., χ is proportional to the squared difference between them. Therefore, the miscibility of polymers can be approximately predicted. For example, the closer the solubility parameters of the two polymers are the higher the possibility that the two polymers are miscible. Miscibility between polymers tends to decrease as their molecular weights increases.

Thus in addition to the experimental determinations, the miscibility between polymers can be predicted simply based on the Flory-Huggins interaction parameters, or even more simply, based the solubility parameters of the components. However, because of the molecular weight effect, close solubility parameters do not necessarily guarantee miscibility.

It should be understood that a mixture of polymers needs only to meet one of the definitions provided herein to be miscible. Furthermore, a mixture of polymers may become a miscible blend upon incorporation of an active agent.

The polymers in the miscible polymer blends can be crosslinked or not. Similarly, the blended polymers can be crosslinked or not. Such crosslinking can be carried out by one of skill in the art after blending using standard techniques.

Certain embodiments of the present invention include segmented polymers. As used herein, a “segmented polymer” is composed of multiple blocks, each of which can separate into the phase that is primarily composed of itself. As used herein, a “hard” segment or “hard” phase of a polymer is one that is either crystalline at use temperature or amorphous with a glass transition temperature above use temperature (i.e., glassy), and a “soft” segment or “soft” phase of a polymer is one that is amorphous with a glass transition temperature below use temperature (i.e., rubbery). Herein, a “segment” refers to the chemical formulation and “phase” refers to the morphology, which primarily includes the corresponding segment (e.g., hard segments form a hard phase), but can include some of the other segment (e.g., soft segments in a hard phase).

As used herein, a “hard” phase of a blend includes primarily a segmented polymer's hard segment and optionally at least part of a second polymer blended therein. Similarly, a “soft” phase of a blend includes predominantly a segmented polymer's soft segment and optionally at least part of a second polymer blended therein. Preferably, miscible blends of polymers of the present invention include blends of segmented polymers' soft segments.

When referring to the solubility parameter of a segmented polymer, “segment” is used and when referring to Tg of a segmented polymer, “phase” is used. Thus, the solubility parameter, which is typically a calculated value for segmented polymers, refers to the hard and/or soft segment of an individual polymer molecule, whereas the Tg, which is typically a measured value, refers to the hard and/or soft phase of the bulk polymer.

Active agents can be incorporated into one or more layers of the systems of the present invention, whether they are miscible polymer blend layers, or layers of immiscible mixtures of polymers, layers containing only one polymer, or layers containing just one or more active agents.

The types and amounts of polymers and active agents are typically selected to form a system having a preselected dissolution time through a preselected critical dimension of the miscible polymer blend layer or layers. Glass transition temperatures, swellabilities, and solubility parameters of the polymers can be used in guiding one of skill in the art to select an appropriate combination of components in an active agent delivery system, whether the active agent is incorporated into the miscible polymer blend or not. Solubility parameters are generally useful for determining miscibility of the polymers and matching the solubility of the active agent to that of the miscible polymer blend. Glass transition temperatures and/or swellabilities are generally useful for tuning the dissolution time (or rate) of the active agent. These concepts are discussed in greater detail below. One or Two Active Agents in a Single Polymer Layer

For embodiments of systems of the present invention in which one or two active agents are present in a layer containing only one polymer, known theories of drug loading apply. For example, if there are two active agents present, then the active agent that is to be delivered faster is the one that is better matched to the solubility of the polymer. One Active Agent in a Miscible Polymer Blend Layer For a miscible polymer blend layer that includes one active agent therein, the theories used are generally coupled to the molecular weight and relative hydrophilicity/hydrophobicity of the active agent. These theories and examples described in the following copending applications of Applicants' Assignee can be applied: ACTIVE AGENT DELIVERY SYSTEMS, MEDICAL DEVICES, AND METHODS, U.S. patent application Ser. No. 10/640,853, filed on Aug. 13, 2003 (published as US 2004/0086569A1 on May 6, 2004); ACTIVE AGENT DELIVERY SYSTEM INCLUDING A HYDROPHOBIC CELLULOSE DERIVATIVE, MEDICAL DEVICE, AND METHOD, U.S. patent application Ser. No. 10/640,714, filed on Aug. 13, 2003 (published as US 2004/0115273A1 on Jun. 17, 2004); ACTIVE AGENT DELIVERY SYSTEM INCLUDING A POLYURETHANE, MEDICAL DEVICE, AND METHOD, U.S. patent application Ser. No. 10/640,823, filed on Aug. 13, 2003 (published as US 2004/0033251A1 on Feb. 19, 2004); ACTIVE AGENT DELIVERY SYSTEM INCLUDING A POLY(ETHYLENE-CO-(METH)ACRYLATE), MEDICAL DEVICE, AND METHOD, U.S. patent application Ser. No. 10/640,702, filed on Aug. 13, 2003 (published as US 2004/0047911A1 on Mar. 11, 2004); and ACTIVE AGENT DELIVERY SYSTEM INCLUDING A HYDROPHILIC POLYMER, MEDICAL DEVICE, AND METHOD, U.S. patent application Ser. No. 10/640,713, filed on Aug. 13, 2003 (published as US 2004/0127978A1 on Jul. 1, 2004).

For preferred active agent delivery systems of the present invention, the active agent is typically matched to the solubility of the miscible portion of the polymer blend. Thus, for embodiments of the invention in which the active agents are hydrophilic, preferably at least one miscible polymer of the miscible polymer blend is hydrophilic. For embodiments of the invention in which the active agents are hydrophobic, preferably at least one miscible polymer of the miscible polymer blend is hydrophobic. However, this is not necessarily required, and it may be undesirable to have a hydrophilic polymer in a delivery system for a low molecular weight hydrophilic active agent because of the potential for swelling of the polymers by water and the loss of controlled delivery of the active agent.

As used herein, in this context (in the context of the polymer of the blend), the term “hydrophilic” refers to a material that will increase in volume by more than 10% or in weight by at least 10%, whichever comes first, when swollen by water at body temperature (i.e., about 37° C.). As used herein, in this context (in the context of the polymer of the blend), the term “hydrophobic” refers to a material that will not increase in volume by more than 10% or in weight by more than 10%, whichever comes first, when swollen by water at body temperature (i.e., about 37° C.).

As used herein, in this context (in the context of the active agent), the term “hydrophilic” refers to an active agent that has a solubility in water of more than 200 micrograms per milliliter. As used herein, in this context (in the context of the active agent), the term “hydrophobic” refers to an active agent that has a solubility in water of no more than 200 micrograms per milliliter.

As the size of the active agent gets sufficiently large, diffusion through the polymer is affected. Thus, active agents can be categorized based on molecular weights and polymers can be selected depending on the range of molecular weights of the active agents.

For certain preferred active agent delivery systems of the present invention, the active agents have a molecular weight of greater than about 1200 g/mol. For certain other preferred active agent delivery systems of the present invention, the active agents have a molecular weight of no greater than (i.e., less than or equal to) about 1200 g/mol. For even more preferred embodiments, active agents of a molecular weight no greater than about 800 g/mol are desired.

Once the active agents and the format for delivery (e.g., time/rate and critical dimension) are selected, one of skill in the art can utilize the teachings of the present invention to select the appropriate combination of at least two polymers.

The types and amounts of polymers and active agents are typically selected to form a system having a preselected dissolution time (t) through a preselected critical dimension (x) of a layer or layers of a miscible polymer blend. This involves selecting at least two polymers to provide a target diffusivity, which is directly proportional to the critical dimension squared divided by the time (x²/t), for a given active agent.

In refining the selection of the polymers for the desired active agent, the desired dissolution time (or rate), and the desired critical dimension, the parameters that can be considered when selecting the polymers for the desired active agent include glass transition temperatures of the polymers, swellabilities of the polymers, solubility parameters of the polymers, and solubility parameters of the active agents. These can be used in guiding one of skill in the art to select an appropriate combination of components in an active agent delivery system, whether the active agent is incorporated into the miscible polymer blend or not.

For enhancing the versatility of a permeation-controlled delivery system, for example, preferably the polymers for a miscible polymer blend layer are selected such that at least one of the following relationships is true: (1) the difference between the solubility parameter of the active agent and at least one solubility parameter of at least one polymer is no greater than about 10 J^{1/2}/cm^{3/2} (preferably, no greater than about 5 J^{1/2}/cm^{3/2}, and more preferably, no greater than about 3 J^{1/2}/cm^{3/2}); and (2) the difference between at least one solubility parameter of each of at least two polymers is no greater than about 3 J^{1/2}/cm^{3/2} (preferably, no greater than about 3 J^{1/2}/cm^{3/2}). More preferably, both relationships are true. Most preferably, both relationships are true for all polymers of the blend.

Typically, a compound has only one solubility parameter, although certain polymers, such as segmented copolymers and block copolymers, for example, can have more than one solubility parameter. Solubility parameters can be measured or they are calculated using an average of the values calculated using the Hoy Method and the Hoftyzer-van Krevelen Method (chemical group contribution methods), as disclosed in D. W. van Krevelen, Properties of Polymers, 3^{rd} Edition, Elsevier, Amsterdam. To calculate these values, the volume of each chemical is needed, which can be calculated using the Fedors Method, disclosed in the same reference.

Solubility parameters can also be calculated with computer simulations, for example, molecular dynamics simulation and Monte Carlo simulation. Specifically, the molecular dynamics simulation can be conducted with Accelrys Materials Studio, Accelrys Inc., San Diego, Calif. The computer simulations can be used to directly calculate the Flory-Huggins parameter.

Examples of solubility parameters for various polymers and active agents are shown in Table 1.

TABLE 1

| | Solubility | | | | | |
|---|---|---|---|---|---|---|
| | parameter | | | | | |
| Polymers | (J^{1/2}/cm^{3/2}) | Source | Notes | Tg (° C.) | Notes | Source |
| polyethylene | 16.45 | 1 | | −94 | | 1 |
| polypropylene | 17.8 | 1 | | −10 | Isotactic | 1 |
| polyisobutylene | 16.3 | 1 | | −71.5 | | 1 |
| polystyrene | 18.2 | 1 | | 102.5 | Atactic | 1 |
| poly(vinyl chloride) | 20.65 | 1 | | 84 | | 1 |
| poly(vinyl bromide) | 19.4 | 1 | | | | |
| poly(vinylidene chloride) | 22.65 | 1 | | −1.5 | | 2 |
| poly(tetrafluoroethylene) | 12.7 | 1 | | 27.5 | | 1 |
| poly(chloro trifluoroethylene) | 15.45 | 1 | | 45 | | 1 |
| poly(vinyl alcohol) | 27.45 | 1 | | 85 | | 1 |
| poly(vinyl acetate) | 20.85 | 1 | | 28 | | 1 |
| poly(vinyl propionate) | 18 | 1 | | | | |
| poly(methyl acylate) | 20.6 | 1 | | 4.5 | | 1 |
| poly(ethyl acrylate) | 19 | 1 | | −24 | | 1 |
| poly(propyl acrylate) | 18.5 | 1 | | | | |
| poly(butyl acrylate) | 18.3 | 1 | | −56 | | 1 |
| poly(isobutyl acrylate) | 20.15 | 1 | | | | |
| poly(2,2,3,3,4,4,4- | 13.7 | 1 | | | | |
| heptafluorobutyl acrylate) | | | | | | |
| poly(methyl methacrylate) | 22.4 | 1 | | 105 | Atactic | 1 |
| poly(ethyl methacrylate) | 18.45 | 1 | | 65 | | 1 |
| poly(butyl methacrylate) | 18.1 | 1 | | 21 | | 1 |
| poly(isobutyl methacrylate) | 19.15 | 1 | | | | |
| poly(tert-butyl methacrylate) | 17 | 1 | | | | |
| poly(benzyl methacrylate) | 20.3 | 1 | | | | |
| poly(ethoxyethyl | 19.35 | 1 | | | | |
| methacrylate) | | | | | | |
| polyacrylonitrile | 28.55 | 1 | | 117 | Syndiotactic, | 1 |
| polymethacrylonitrile | 21.9 | 1 | | 120 | | 1 |
| poly(alpha-cyanomethyl | 29.2 | 1 | | | | |
| acrylate) | | | | | | |
| polybutadiene | 17.1 | 1 | | −50.5 | Trans 1,4- | 1 |
| | | | | | butadiene | |
| polyisoprene | 18.35 | 1 | | −59 | Trans | 1 |
| polychloroprene | 17.85 | 1 | | | | |
| polyformaldehyde | 21.7 | 1 | | −66.5 | | 1 |
| poly(tetramethylene oxide) | 17.25 | 1 | | −83.5 | | 2 |
| poly(propylene oxide) | 17.85 | 1 | | | | |
| polyepichlorohydrin | 19.2 | 1 | | | | |
| poly(ethylene sulphide) | 18.8 | 1 | | | | |
| poly(styrene sulphide) | 19 | 1 | | | | |
| poly(ethylene terephthalate) | 20.9 | 1 | | 69 | | 1 |
| poly(8-aminocaprylic acid) | 26 | 1 | | | | |
| poly(hexamethylene | 27.8 | 1 | | | | |
| adipamide) | | | | | | |
| polyurethane hard segment | 23.35 | 2 | H-vK, urethane NHCOO = NH + COO. | 10 | | RSA |
| (MDI + BDO) | | | Fedors volume | | | |
| | | | 230 cm³/mol | | | |
| poly(bisphenyl A carbonate) | 22.9 | 2 | H-vK, carbonate OCOO = COO + O; | 140 | | 1 |
| | | | Hoy OCOO = O + COO. | | | |
| | | | Fedors volume 174 cm³/mol | | | |
| cellulose acetate butyrate | 21.8 | 2 | The total numbers of acetyl, | 110 | | TSC |
| (acetyl 29.5 wt- %, butyryl | | | butyryl, and OH has to be 3 | | | |
| 17 wt- %) | | | per repeat unit. It was | | | |
| | | | estimated the wt- % of OH | | | |
| | | | was 1.1 and the molecular | | | |
| | | | weight of the repeat unit was | | | |
| | | | 303 g/mol. Fedors volume | | | |
| | | | 188 cm³/mol | | | |
| phenoxy | 23.2 | 2 | Fedors volume 201 cm³/mol | 95 | | Vendor |
| poly(vinyl pyrrolidone) | 25.1 | 2 | CON = CO + tertiary N. | 175 | | 1 |
| | | | Fedors volume 65 cm³/mol | | | |
| poly(vinyl pyrrolidone) co poly | 21.7 | 2 | CON = CO + tertiary N. | | | |
| (vinyl acetate) (1.3/1 wt) | | | Fedors volume 132 cm³/mol | | | |
| poly(ethylene oxide) | 22.15 | 2 | Fedors volume 36 cm³/mol | −47 | | 2 |
| dexamethasone | 27.25 | 2 | All rings were treated as | | | |
| | | | aliphatic. Hydroxyl groups | | | |
| | | | were not involved in | | | |
| | | | hydrogen bonding. Fedors | | | |
| | | | volume 205 cm³/mol | | | |
| Rosiglitazone maleate | 23.45 | 2 | H-vK, C5NH5 as C6H5*5/6 + tertiary | | | |
| | | | N, CONHCO as 2CO + NH; | | | |
| | | | Hoy, aromatic tertiary | | | |
| | | | N treated as aliphatic tertiary | | | |
| | | | N, CONHOC as CONH + CO. | | | |
| | | | Fedors volume 306 cm³/mol | | | |
| Source for Solubility Parameters: | | | | | | |
| 1. D. W. van Krevelen, Properties of Polymers, 3rd ed., Elsevier, 1990. Table 7.5. Data were the average if there were two values listed in the sources. | | | | | | |
| 2. Average of the calculated values based on Hoftyzer and van Kevelen's (H-vK) method (where the volumes of the chemicals were calculated based on Fedors' method) and Hoy's method. See Chapter 7, D. W. van Krevelen, Properties of Polymers, 3rd ed., Elsevier, 1990, for details of all the calculations, where Table 7.8 was for Hoftyzer | | | | | | |
| # and van Kevelen's method, Table 7.3 for Fedors' method, and Table 7.9 and 7.10 for Hoy's method. | | | | | | |

Source of Tg's (the reported value is the average if there are two values listed in the sources):
1. Table 6.6, J. M. He, W. X. Chen, and X. X. Dong, Polymer Physics, revised version, FuDan University Press, ShangHai, China, 2000. Data were the average if there were two values listed in the sources.
2. Table 6.4, D. W. van Krevelen, Properties of Polymers, 3rd ed., Elsevier, 1990. Data were the average if there were two values listed in the sources.

For delivery systems in which the active agent is hydrophobic, regardless of the molecular weight, polymers are typically selected such that the molar average solubility parameter of the miscible polymer blend is no greater than 28 J^{1/2}/cm^{3/2}(preferably, no greater than 25 J^{1/2}/cm^{3/2}). For delivery systems in which the active agent is hydrophilic, regardless of the molecular weight, polymers are typically selected such that the molar average solubility parameter of the miscible polymer blend is greater than 21 J^{1/2}/cm^{3/2}(preferably, greater than 25 J^{1/2}/cm^{3/2}).

Herein “molar average solubility parameter” means the average of the solubility parameters of the blend components that are miscible with each other and that form the continuous portion of the miscible polymer blend. These are weighted by their molar percentage in the blend, without the active agent incorporated into the polymer blend.

For enhancing the tunability of permeation-controlled dissolution times (rates) for low molecular weight active agents, preferably the polymers can be selected such that the difference between at least one Tg of at least two of the polymers corresponds to a range of diffusivities that includes the target diffusivity.

Alternatively, for enhancing the tunability of permeation-controlled dissolution times (rates) for high molecular weight active agents, preferably the polymers can be selected such that the difference between the swellabilities of at least two of the polymers of the blend corresponds to a range of diffusivities that includes the target diffusivity. The target diffusivity is determined by the preselected time (t) for delivery and the preselected critical dimension (x) of the polymer composition of a layer and is directly proportional to x²/t.

The target diffusivity can be easily measured by dissolution analysis using the following equation (see, for example, Kinam Park edited, Controlled Drug Delivery: Challenges and Strategies, American Chemical Society, Washington, D.C., 1997):

$$D = {( \frac{{M}_{t}}{4 {M}_{∞}} )}^{2} \cdot \frac{π {x}^{2}}{t}$$

wherein D=diffusion coefficient; Mₜ=cumulative release; M∞=total loading of active agent; x=the critical dimension (e.g., thickness of a layer or layers); and t=the dissolution time. This equation is valid during dissolution of up to 60 percent by weight of the initial load of the active agent. Also, blend samples should be in the form of a film.

Generally, at least one polymer has an active agent diffusivity higher than the target diffusivity and at least one polymer has an active agent diffusivity lower than the target diffusivity. The diffusivity of a polymer system can be easily measured by dissolution analysis, which is known to one of skill in the art. The diffusivity of an active agent from each of the individual polymers can be determined by dissolution analysis, but can be estimated by relative Tg's or swellabilities of the major phase of each polymer.

The diffusivity can be correlated to glass transition temperatures of hydrophobic or hydrophilic polymers, which can be used to design a delivery system for low molecular weight active agents (e.g., those having a molecular weight of no greater than about 1200 g/mol). Alternatively, the diffusivity can be correlated to swellabilities of hydrophobic or hydrophilic polymers, which can be used to design a delivery system for high molecular weight polymers (e.g., those having a molecular weight of greater than about 1200 g/mol). This is advantageous because the range of miscible blends can be used to encompass very different dissolution rates for active agents of similar solubility.

The glass transition temperature of a polymer is a well-known parameter, which is typically a measured value. Exemplary values are listed in Table 1. For segmented polymers (e.g., a segmented polyurethane) the Tg refers to the particular phase of the bulk polymer. Typically, for low molecular weight active agents, by selecting relatively low and high Tg polymers that are miscible, the dissolution kinetics of the system can be tuned. This is because a small molecular weight agent (e.g., no greater than about 1200 g/mol) diffuses through a path that is directly correlated with the Tg's, i.e., the free volume of the polymer blend is a linear function of the temperature with slope being greater when the temperature is above Tg.

Preferably, a polymer having at least one relatively high Tg is combined with a polymer having at least one relatively low Tg. By combining such high and low Tg polymers, the active agent delivery system can be tuned for the desired dissolution time of the active agent.

Swellabilities of polymers in water can be easily determined. It should be understood, however, that the swellability results from incorporation of water and not from an elevation in temperature. Typically, for high molecular weight active agents, by selecting relatively low and high swell polymers that are miscible, the dissolution kinetics of the system can be tuned. Swellabilities of polymers are used to design these systems because water needs to diffuse into the polymer blend to increase the free volume for active agents of relatively high molecular weight (e.g., greater than about 1200 g/mol) to diffuse out of the polymeric blend.

Preferably, a polymer having a relatively high swellability is combined with a polymer having a relatively low swellability. By combining such high and low swell polymers, the active agent delivery system can be tuned for the desired dissolution time of the active agent.

Swellabilities of the miscible polymer blends are also used as a factor in determining the combinations of polymers for a particular active agent. For delivery systems in which the active agent has a molecular weight of greater than 1200 g/mol, whether it is hydrophilic or hydrophobic, polymers are selected such that the swellability of the blend is greater than 10% by volume. The swellability of the blend is evaluated without the active agent incorporated therein.

In one embodiment, for an active agent delivery system (having a target diffusivity) that includes an active agent that is hydrophobic and has a molecular weight of no greater than (i.e., less than or equal to) about 1200 g/mol, the miscible polymer blend includes at least two polymers, each with at least one solubility parameter, wherein: the difference between the solubility parameter of the active agent and at least one solubility parameter of at least one of the polymers is no greater than about 10 J^{1/2}/cm^{3/2}, and the difference between at least one solubility parameter of each of at least two polymers is no greater than about 5 J^{1/2}/cm^{3/2}; at least one polymer has an active agent diffusivity higher than the target diffusivity and at least one polymer has an active agent diffusivity lower than the target diffusivity; the molar average solubility parameter of the blend is no greater than 28 J^{1/2}/cm^{3/2} (preferably, no greater than 25 J^{1/2}/cm^{3/2}) and the swellability of the blend is no greater than 10% by volume.

In one embodiment, for an active agent delivery system (having a target diffusivity) that includes an active agent that is hydrophobic and has a molecular weight of no greater than (i.e., less than or equal to) about 1200 g/mol, the miscible polymer blend includes at least two polymers, wherein: the difference between the solubility parameter of the active agent and at least one solubility parameter of at least one of the polymers is no greater than about 10 J^{1/2}/cm^{3/2}, and the difference between at least one solubility parameter of each of at least two polymers is no greater than about 5 J^{1/2}/cm^{3/2}; at least one polymer has an active agent diffusivity higher than the target diffusivity and at least one polymer has an active agent diffusivity lower than the target diffusivity; the molar average solubility parameter of the blend is greater than 21 J^{1/2}/cm^{3/2} (preferably, greater than 25 J^{1/2}/cm^{3/2}); and the swellability of the blend is no greater than 10% by volume.

In one embodiment, for an active agent delivery system (having a target diffusivity) that includes an active agent that is hydrophobic and has a molecular weight of greater than about 1200 g/mol, the miscible polymer blend includes at least two polymers, wherein: the difference between the solubility parameter of the active agent and at least one solubility parameter of at least one of the polymers is no greater than about 10 J^{1/2}/cm^{3/2}, and the difference between at least one solubility parameter of each of at least two polymers is no greater than about 5 J^{1/2}/cm^{3/2}; at least one polymer has an active agent diffusivity higher than the target diffusivity and at least one polymer has an active agent diffusivity lower than the target diffusivity; the molar average solubility parameter of the blend is no greater than 28 J^{1/2}/cm^{3/2} (preferably, no greater than 25 J^{1/2}/cm^{3/2}); and the swellability of the blend is greater than 10% by volume.

In one embodiment, for an active agent delivery system (having a target diffusivity) that includes an active agent that is hydrophobic and has a molecular weight of greater than about 1200 g/mol, the miscible polymer blend includes at least two polymers, wherein: the difference between the solubility parameter of the active agent and at least one solubility parameter of at least one of the polymers is no greater than about 10 J^{1/2}/cm^{3/2}, and the difference between at least one solubility parameter of each of at least two polymers is no greater than about 5 J^{1/2}/cm^{3/2}; at least one polymer has an active agent diffusivity higher than the target diffusivity and at least one polymer has an active agent diffusivity lower than the target diffusivity; the molar average solubility parameter of the blend is greater than 21 J^{1/2}/cm^{3/2} (preferably, greater than 25 J^{1/2}/cm^{3/2}); and the swellability of the blend is greater than 10% by volume.

Two or More Active Agents in One Miscible Polymer Blend Layer

For situations in which there are two or more active agents in a layer of two or more miscible polymers, theories similar to those described above with respect to one active agent in a layer that includes a miscible polymer blend layer can be used. These theories are described in greater detail in Applicants' Assignee's copending application entitled ACTIVE AGENT DELIVERY SYSTEMS INCLUDING A SINGLE LAYER OF A MISCIBLE POLYMER BLEND, MEDICAL DEVICES, AND METHODS, U.S. patent application Ser. No. 60/495,022, filed on Aug. 13, 2003.

In sum, the two or more active agents are selected such that the permeability of the active agent that is to be released faster is greater than the permeability of the other one or more active agents. In this context, the “permeability” of an active agent is its diffusivity times its solubility.

Preferably, the two or more active agents are selected such that the difference between the solubility parameter of the active agent that is to be released faster and to be present in a greater amount (i.e., greater load) and the molar average solubility parameter of the at least two miscible polymers is smaller than the differences between the solubility parameter of each of the other one or more active agents and the molar average solubility parameter of the at least two miscible polymers.

For such systems, it is preferable that the active agents are at or below the solubility limit of the miscible polymer blend. That is, the solubility parameters of each of the active agents and at least one polymer of the miscible polymer blend are matched to maximize the level of loading while decreasing the tendency for delivery by a porosity mechanism. Although not wishing to be bound by theory, it is believed that because of this mechanism the active agent delivery systems of the present invention have a significant level of tunability.

Active Agents

As used herein, an “active agent” is one that produces a local or systemic effect in a subject (e.g., an animal). Typically, it is a pharmacologically active substance. The term is used to encompass any substance intended for use in the diagnosis, cure, mitigation, treatment, or prevention of disease or in the enhancement of desirable physical or mental development and conditions in a subject. The term “subject” used herein is taken to include humans, sheep, horses, cattle, pigs, dogs, cats, rats, mice, birds, reptiles, fish, insects, arachnids, protists (e.g., protozoa), and prokaryotic bacteria. Preferably, the subject is a human or other mammal.

Active agents can be synthetic or naturally occurring and include, without limitation, organic and inorganic chemical agents, polypeptides (which is used herein to encompass a polymer of L- or D-amino acids of any length including peptides, oligopeptides, proteins, enzymes, hormones, etc.), polynucleotides (which is used herein to encompass a polymer of nucleic acids of any length including oligonucleotides, single- and double-stranded DNA, single- and double-stranded RNA, DNA/RNA chimeras, etc.), saccharides (e.g., mono-, di-, poly-saccharides, and mucopolysaccharides), vitamins, viral agents, and other living material, radionuclides, and the like. Examples include antithrombogenic and anticoagulant agents such as heparin, coumadin, protamine, and hirudin; antimicrobial agents such as antibiotics; antineoplastic agents and anti-proliferative agents such as etoposide, podophylotoxin; antiplatelet agents including aspirin and dipyridamole; antimitotics (cytotoxic agents) and antimetabolites such as methotrexate, colchicine, azathioprine, vincristine, vinblastine, fluorouracil, adriamycin, and mutamycinnucleic acids; antidiabetic such as rosiglitazone maleate; and anti-inflammatory agents. Anti-inflammatory agents for use in the present invention include glucocorticoids, their salts, and derivatives thereof, such as cortisol, cortisone, fludrocortisone, Prednisone, Prednisolone, 6α-methylprednisolone, triamcinolone, betamethasone, dexamethasone, beclomethasone, aclomethasone, amcinonide, clebethasol and clocortolone. Preferably, the active agent is not heparin.

Certain preferred systems include an active agent selected from the group consisting of indomethacin, sulindac, diclofenal, etodolac, meclofenate, mefenamic acid, nambunetone, piroxicam, phenylgutazone, meloxicam, dexamethoasone, betamethasone, dipropionate, diflorsasone diacetate, clobetasol propionate, galobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, beta-estradiol, rosiglitazone, troglitazone, pioglitazone, S-nitrosoglutathione, gliotoxin G, panepoxydone, cycloepoxydon tepoxalin, curcumin, a proteasome inhibitor (e.g., bortezomib, dipeptide boronic acid, lactacystin, bisphosphonate, zolendronate, epoxomicin), antisense c-myc, celocoxib, valdecoxib, and combinations thereof. These active agents are typically selected to be the faster active agent released. Typically, it is also the first one initially released, although this is not a necessary requirement. Herein, this active agent is referred to as the first active agent.

Certain preferred systems include an active agent selected from the group consisting of podophyllotoxin, mycophenolic acid, teniposide, etoposide, trans-retinoic acids, 9-cis retinoic acid, 13-cis retinoic acid, rapamycin, a rapalog (e.g., Everolimus, ABT-578), camptothecin, irinotecan, topotecan, tacromilus, mithramycin, mitobronitol, thiotepa, treosulfan, estramusting, chlormethine, carmustine, lomustine, busultan, mephalan, chlorambucil, ifosfamide, cyclophosphamide, doxorubicin, epirubicin, aclarubicin, daunorubicin, mitosanthrone, bleomycin, cepecitabine, cytarabine, fludarabine, cladribine, gemtabine, 5-fluorouracil, mercaptopurine, tioguanine, vinblastine, vincristine, vindesine, vinorelbine, amsacrine, bexarotene, crisantaspase, decarbasine, hydrosycarbamide, pentostatin, carboplatin, cisplatin, oxiplatin, procarbazine, paclitaxel, docetaxel, epothilone A, epothilone B, epothilone D, baxiliximab, daclizumab, interferon alpha, interferon beta, maytansine, and combinations thereof. These active agents are typically selected to be released at a slower rate than that of the first active agent, and/or after the start of release of the first active agent, for example. Generally, the concept is to release at least two active agents spread apart in time.

In certain preferred systems, one active agent is sulfasalzine, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is indomethacin, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is ascomycin, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is leflunomide, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is dexamethasone, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is piroxicam, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is beclomethasone dipropionate, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is S-nitrosoglutathione, and at least one active agent is selected from the group consisting of podophyllotoxin, mycophenolic acid, teniposide, etoposide, camptothecin, irinotecan, topotecan, mithramycin, and combinations thereof.

In certain preferred systems, one active agent is rosiglitazone, and at least one active agent is selected from the group consisting of trans-retinoic acids, 9-cis retinoic acid, 13-cis retinoic acid, etoposide, mycophenolic acid, podophyllotoxin, teniposide, camptothecin, irinotecan, topotecan, mithranycin, and combinations thereof.

In certain preferred systems, one active agent is troglitazone, and at least one active agent is selected from the group consisting of trans-retinoic acids, 9-cis retinoic acid, 13-cis retinoic acid, etoposide, mycophenolic acid, podophyllotoxin, teniposide, camptothecin, irinotecan, topotecan, mithranycin, and combinations thereof.

In certain preferred systems, one active agent is pioglitazone, and at least one active agent is selected from the group consisting of trans-retinoic acids, 9-cis retinoic acid, 13-cis retinoic acid, etoposide, mycophenolic acid, podophyllotoxin, teniposide, camptothecin, irinotecan, topotecan, mithranycin, and combinations thereof.

Typically, the amount of active agents within an active agent delivery system of the present invention is determined by the amount to be delivered and the time period over which it is to be delivered. Other factors can also contribute to the level of active agent present, including, for example, the ability of the composition to form a uniform film on a substrate.

Preferably, each active agent is present within (i.e., incorporated within) any one layer in an amount of at least about 0.1 weight percent (wt-%), more preferably, at least about 1 wt-%, and even more preferably, at least about 5 wt-%, based on the total weight of the layer. Preferably, each active agent is present within a layer in an amount of no greater than about 80 wt-%, more preferably, no greater than about 50 wt-%, and most preferably, no greater than about 30 wt-%, based on the total weight of the layer, although any one layer can include one or more active agents alone. For certain preferred embodiments, the amount of each active agent will be at or below its solubility limit in the miscible polymer blend.

Medical Devices and Methods

The active agent delivery systems of the present invention can be in the form of coatings on substrates (e.g., open or closed cell foams, woven or nonwoven materials), devices (e.g., stents, stent grafts, catheters, shunts, balloons, etc.), films (which can be free-standing as in a patch, for example), shaped objects (e.g., microspheres, beads, rods, fibers, or other shaped objects), wound packing materials, etc.

In the active agent systems of the present invention, the active agents pass through a miscible polymer blend having a “critical” dimension. This critical dimension is along the net diffusion path of the active agent and is preferably no greater than about 1000 micrometers (i.e., microns), although for shaped objects it can be up to about 10,000 microns.

For embodiments in which the miscible polymer blends form coatings or free-standing films (both generically referred to herein as “films”), the critical dimension is the thickness of the film and is preferably no greater than about 1000 microns, more preferably no greater than about 500 microns, and most preferably no greater than about 100 microns. A film can be as thin as desired (e.g., 1 nanometer), but are preferably no thinner than about 10 nanometers, more preferably no thinner than about 100 nanometers. Generally, the minimum film thickness is determined by the volume that is needed to hold the required dose of active agent and is typically only limited by the process used to form the materials. For all embodiments herein, the thickness of the film does not have to be constant or uniform. Furthermore, the thickness of the film can be used to tune the duration of time over which the active agent is released.

For embodiments in which the miscible polymer blends form shaped objects (e.g., microspheres, beads, rods, fibers, or other shaped objects), the critical dimension of the object (e.g., the diameter of a microsphere or rod) is preferably no greater than about 10,000 microns, preferably no greater than about 1000 microns, even more preferably no greater than about 500 microns, and most preferably no greater than about 100 microns. The objects can be as small as desired (e.g., 10 nanometers for the critical dimension). Preferably, the critical dimension is no less than about 100 microns, and more preferably no less than about 500 nanometers.

In one embodiment, the present invention provides a medical device characterized by a substrate surface overlayed with a polymeric top coat layer that includes a miscible polymer blend, preferably with a polymeric undercoat (primer) layer. When the device is in use, the miscible polymer blend is in contact with a bodily fluid, organ, or tissue of a subject.

The invention is not limited by the nature of the medical device; rather, any medical device can include the polymeric coating layer that includes the miscible polymer blend. Thus, as used herein, the term “medical device” refers generally to any device that has surfaces that can, in the ordinary course of their use and operation, contact bodily tissue, organs or fluids such as blood. Examples of medical devices include, without limitation, stents, stent guides, anastomotic connectors, leads, needles, guide wires, catheters, sensors, surgical instruments, angioplasty balloons, wound drains, shunts, tubing, urethral inserts, pellets, implants, pumps, vascular grafts, valves, pacemakers, and the like. A medical device can be an extracorporeal device, such as a device used during surgery, which includes, for example, a blood oxygenator, blood pump, blood sensor, or tubing used to carry blood, and the like, which contact blood which is then returned to the subject. A medical device can likewise be an implantable device such as a vascular graft, stent, stent graft, anastomotic connector, electrical stimulation lead, heart valve, orthopedic device, catheter, shunt, sensor, replacement device for nucleus pulposus, cochlear or middle ear implant, intraocular lens, and the like. Implantable devices include transcutaneous devices such as drug injection ports and the like.

In general, preferred materials used to fabricate the medical device of the invention are biomaterials. A “biomaterial” is a material that is intended for implantation in the human body and/or contact with bodily fluids, tissues, organs and the like, and that has the physical properties such as strength, elasticity, permeability and flexibility required to function for the intended purpose. For implantable devices in particular, the materials used are preferably biocompatible materials, i.e., materials that are not overly toxic to cells or tissue and do not cause undue harm to the body.

The invention is not limited by the nature of the substrate surface for embodiments in which the miscible polymer blends form polymeric coatings. For example, the substrate surface can be composed of ceramic, glass, metal, polymer, or any combination thereof. In embodiments having a metal substrate surface, the metal is typically iron, nickel, gold, cobalt, copper, chrome, molybdenum, titanium, tantalum, aluminum, silver, platinum, carbon, and alloys thereof. A preferred metal is stainless steel, a nickel titanium alloy, such as NITINOL, or a cobalt chrome alloy, such as NP₃₅N.

A polymeric coating that includes a miscible polymer blend can adhere to a substrate surface by either covalent or non-covalent interactions. Non-covalent interactions include ionic interactions, hydrogen bonding, dipole interactions, hydrophobic interactions and van der Waals interactions, for example.

Preferably, the substrate surface is not activated or functionalized prior to application of the miscible polymer blend coating, although in some embodiments pretreatment of the substrate surface may be desirable to promote adhesion. For example, a polymeric undercoat layer (i.e., primer) can be used to enhance adhesion of the polymeric coating to the substrate surface. Suitable polymeric undercoat layers are disclosed in Applicants' Assignee's copending U.S. Provisional Application Ser. No. 60/403,479, filed on Aug. 13, 2002; U.S. patent application Ser. No. 10/640,701, filed on Aug. 13, 2003 (published as US 2004/0039437 A1 on Feb. 26, 2004); and PCT International Patent Application No. PCT/US 03/25463, filed Aug. 13, 2003 (published as WO 2004/014453A1 on Feb. 19, 2004), all of which are entitled MEDICAL DEVICE EXHIBITING IMPROVED ADHESION BETWEEN POLYMERIC COATING AND SUBSTRATE. A particularly preferred undercoat layer disclosed therein consists essentially of a polyurethane. Such a preferred undercoat layer includes a polymer blend that contains polymers other than polyurethane but only in amounts so small that they do not appreciably affect the durometer, durability, adhesive properties, structural integrity and elasticity of the undercoat layer compared to an undercoat layer that is exclusively polyurethane.

When a stent or other vascular prosthesis is implanted into a subject, restenosis is often observed during the period beginning shortly after injury to about four to six months later. Thus, for embodiments of the invention that include stents, the generalized dissolution rates contemplated are such that the active agent should ideally start to be released immediately after the prosthesis is secured to the lumen wall to lessen cell proliferation. The active agent should then continue to dissolute for up to about four to six months in total.

The invention is not limited by the process used to apply the polymer blends to a substrate surface to form a coating. Examples of suitable coating processes include solution processes, powder coating, melt extrusion, or vapor deposition.

A preferred method is solution coating. For solution coating processes, examples of solution processes include spray coating, dip coating, and spin coating. Typical solvents for use in a solution process include tetrahydrofuran (THF), methanol, ethanol, ethylacetate, dimethylformamide (DMF), dimethyacetamide (DMA), dimethylsulfoxide (DMSO), dioxane, N-methyl pyrollidone, chloroform, hexane, heptane, cylcohexane, toluene, formic acid, acetic acid, and/or dichloromethane. Single coats or multiple thin coats can be applied.

Similarly, the invention is not limited by the process used to form the miscible polymer blends into shaped objects. Such methods would depend on the type of shaped object. Examples of suitable processes include extrusion, molding, micromachining, emulsion polymerization methods, electrospray methods, the reflow method described in Applicants' Assignee's copending U.S. Provisional Application Ser. No. 60/403,479, filed on Aug. 13, 2002; U.S. patent application Ser. No. 10/640,701, filed on Aug. 13, 2003 (published as US 2004/0039437 A1 on Feb. 26, 2004); and PCT International Patent Application No. PCT/US 03/25463, filed Aug. 13, 2003 (published as WO 2004/014453A1 on Feb. 19, 2004), all of which are entitled MEDICAL DEVICE EXHIBITING IMPROVED ADHESION BETWEEN POLYMERIC COATING AND SUBSTRATE, etc.

### EXAMPLES

Objects and advantages of this invention are further illustrated by the following examples, but the particular materials and amounts thereof recited in these examples, as well as other conditions and details, should not be construed to unduly limit this invention.

To demonstrate the concept of dual active agent release from coated stent, mycophenolic acid (MA or MPA), podophyllotoxin (Podo) and Etoposide (EP) were selected as anti-proliferative agents and sulfasalazine was selected as an anti-inflammatory agent. Using these active agents, three dual release systems were developed: (1) mycophenolic acid/sulfasalazine (SF), (2) podophyllotoxin/sulfasalazine, and (3) etoposide/sulfasalazine. Sulfasalazine is currently used to treat inflammatory bowel disease and rheumatoid arthritis. It has been found to have numerous biologic effects including immunosuppressive and modulatory actions on lymphocytes and leucocyte function. Sulfasalzine inhibits IL-2 synthesis and IL-1 production in lymphocytes. Sulfasalazine also acts as a potent inhibitor of NF-kB by inhibiting IkB phosphorylation, thereby preventing translocation into the nucleus and decreasing adhesion molecules expression. On the other hand, mycophenolic acid is an immunosuppressant and an inhibitor of the de novo pathway of purine sysnthesis and is a highly selective inhibitor of lymphocyte proliferation. Mycophenolic acid also inhibits SMC and EC in physiological achievable concentrations. Podophyllotoxin is anti-mitotic glucoside and has effects on SMC that are undergoing cell division. Etoposide (EP) is an analog of podophyllotoxin. It acts on the DNA phase of the cell division.

### Example 1

Stainless steel coronary S7 stents (manufactured by Medtronic AVE) were ultrasonically cleaned with isopropanol (IPA) for 30 minutes and allowed to dry thoroughly. The stents were then sprayed with a 0.25% solution of TECOPLAST (TP) polyurethane (Thermedics Polymer) in THF as an initial primer. The stents were then heat-treated at 215-220° C. for 5-15 minutes to create better adhesion between metal and polymer interface. Next, each stent was sprayed with 1% solution of mycophenolic acid (Sigma-Aldrich) in TECOPLAST polyurethane (25 wt-% loading of active agent) using tetrahydrofuran (THF) as solvent. This represents the inner layer with a target coating of 400 micrograms (μg) +/−10% and a thickness of approximately 4 micrometers (μm). The stent was then vacuum-dried at 45° C. overnight and then weighed. After weighing, a thin coating of TECOPLAST polyurethane (1% solution in THF) was sprayed over the first or inner layer to form a barrier. This barrier formed the middle layer that can further slow down the release of mycophenolic acid (MA). The target weight for the middle barrier layer was roughly 100 μg+/−10% or 1 to 2 μm in thickness. After the middle layer barrier was formed, the stent was again vacuum-dried in an oven at 45° C. overnight and then weighed. Next, the stent was sprayed with a 1% solution of 30% sulfasalazine (SF, from Sigma-Aldrich) in 60/40% blend of TECOPLAST/PEVA (Dupont 40W) in THF to form the outer layer that contains the inflammatory active agent, which was designed to release first and at a faster rate. The target for the outer layer/coat was 600 μg +/−10% or roughly 6 μm in thickness. The stent was again vacuum-dried in an oven at 45° C. overnight and weighed to determine the theoretical content of the active agents.

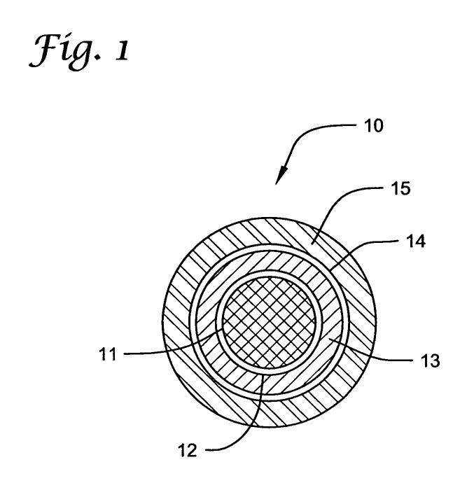

The design of this system 10 is shown in FIG. 1, wherein the stent wire 11 is coated with a primer layer 12, which is coated with an inner layer 13 of a TECOPLAST polyurethane with mycophenolic acid therein. Over the inner layer 13 is a barrier layer 14 of a TECOPLAST polyurethane, which is coated with an outer layer 15 of a TECOPLAST/PEVA blend with sulfasalazine therein. The primer layer 12 can include, for example, about 25 micrograms (μg) to about 50 micrograms of the primer.

The in vitro elution kinetics of dual release was carried out in PBS and at 37° C. The stent was crimped on a stent delivery system and then expanded. After expansion, the physical aspects of the stent were noted prior to placing the stent inside a vial containing 3 milliliters (ml) of PBS. The vial was placed in a shaker at 37° C. and at certain time intervals; the whole solution (3 ml) was removed and replaced with fresh PBS. The amount of each active agent in each dual release system was determined by UV-Vis spectrophotometer using wavelengths of 250 nanometers (nm) for mycophenolic acid and 359 nm for sulfasalazine and solving simultaneous equations of active agent mixtures.

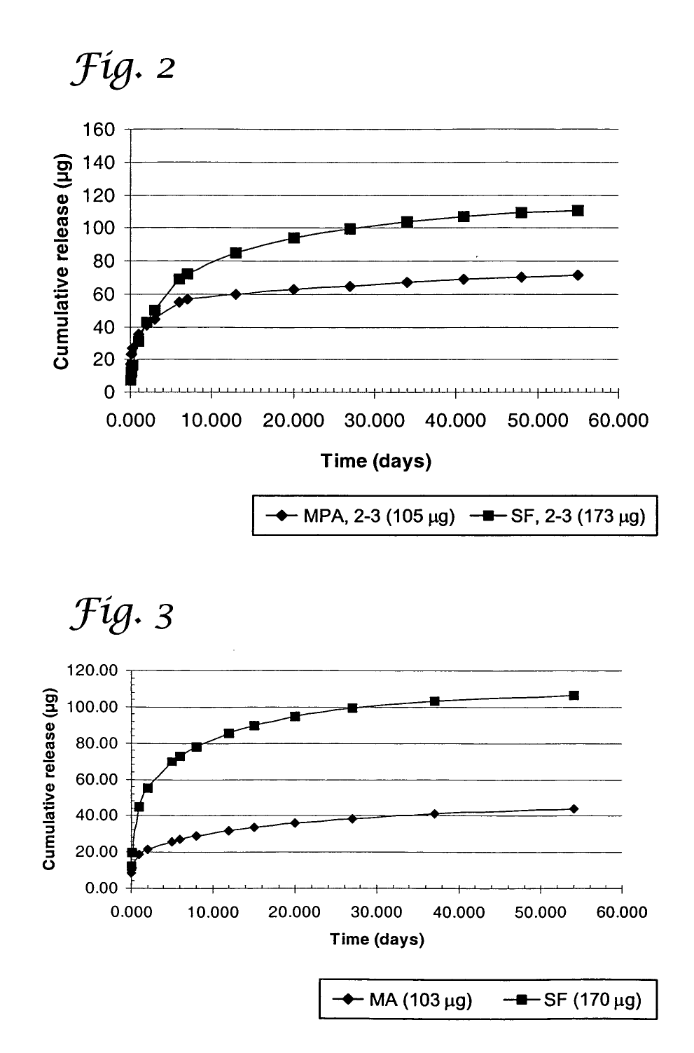

FIG. 2 shows the release kinetics of mycophenolic acid and sulfasalazine of the system in FIG. 1 where there was not a middle barrier layer while FIG. 3 shows that by placing a rate limiting barrier in the middle, the release kinetics of the active agent in the inner layer (mycophenolic acid in this case) can be slowed down significantly.

### Example 2

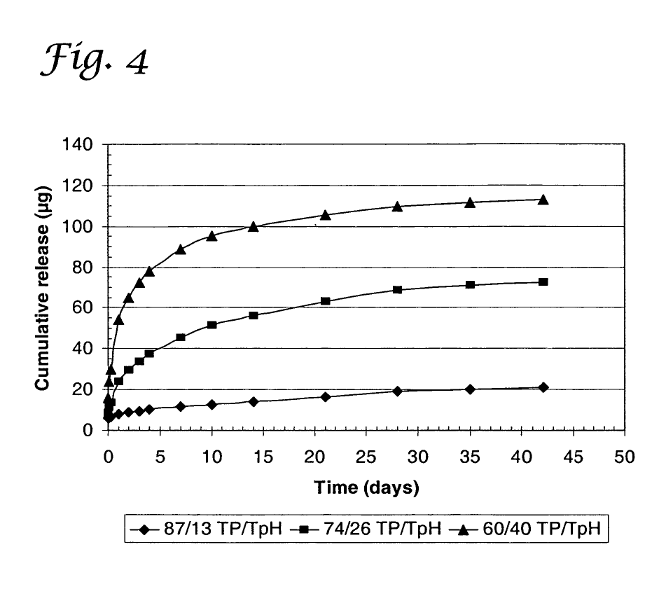

Using the same procedure as in Example 1, similar dual active agent-coated stents were fabricated except in this case the blend of the outer layer of TECOPLAST/PEVA was replaced with TECOPLAST/TECOPHILIC polyurethanes (Thermedics Polymer). The release of sulfasalazine from a blend of TECOPLAST (TP) and TECOPHILIC (TpH) polyurethanes for use as an outer layer is shown in FIG. 4. The release rate of sulfasalazine increased as the percentage of TECOPHILIC polyurethane in the blend increased.

### Example 3

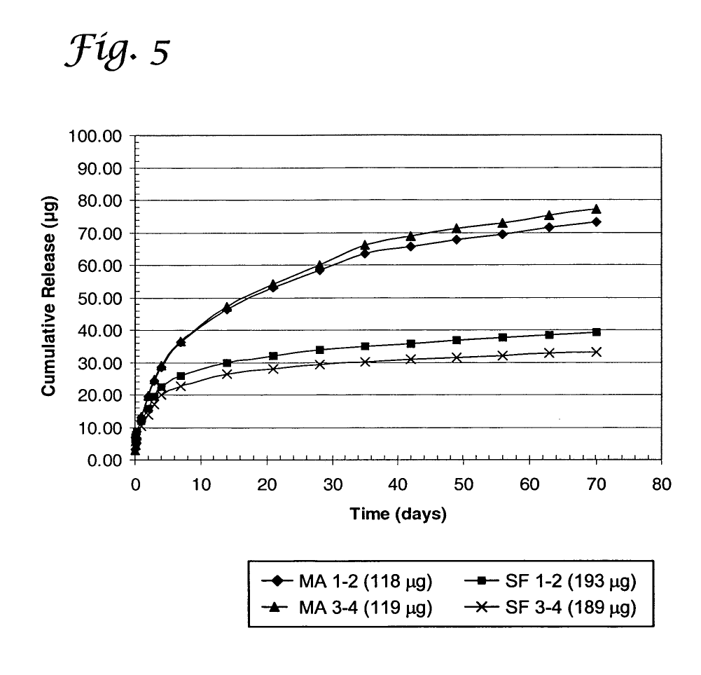

Coated stents were fabricated as described in Example 1 and FIG. 1, except the design consisted of no middle barrier and its inner layer consisted of a blend of 80% TECOPLAST/20% TECOTHANE 75D or just TECOPLAST alone, and the outer layer consisted of a blend of 70% TECOPLAST/20% TECOTHANE 75D. This shows that polymer blends can be used to change the release characteristics of active agents. The release characteristics of this system are shown in FIG. 5. In FIG. 5, MA1-2 is the average cumulative release of mycophenolic acid from samples 1 and 2; SF1-2 is the average cumulative release of sulfasalazine from samples 1 and 2. For samples 1 and 2, the inner layer contains 30% of mycophenolic acid in TECOPLAST polyurethane and the outer layer contains 35% of sulfasalazine in a blend of 70% TECOPLAST and 30% of TECOTHANE 75D polyurethanes. Similarly, MA3-4 is the average cumulative release of mycophenolic acid from samples 3 and 4 and SF3-4 is the average cumulative release of sulfasalazine from samples 3 and 4. The only difference in these samples (3 and 4) compared to samples 1 and 2 is that the inner layer is a blend of 80% TECOPLAST and 20% TECOTHANE 75D polyurethanes.

### Example 4

Primed stents were prepared as in Example 1. Next, each stent was sprayed with 1% solution of podophyllotoxin (podo, Sigma-Aldrich) in TECOPLAST (TP) or TECOTHANE (TH) polyurethane (10 or 20% loading of active agent) using THF as solvent. This represents the inner layer with a target coating of 400 μg+/−10% and a thickness of approximately 4 μm. The stent was then vacuum-dried at 45° C. overnight and then weighed. Next, the stent was sprayed with a 1% solution of 30% sulfasalazine (SF, Sigma-Aldrich) in 60/40% TECOPLAST/PEVA (Dupont 40W) in THF to form the outer layer. The target for the outer layer/coat was 600 μg+/−10% or roughly 6 μm in thickness. The stent was again vacuum-dried in an oven at 45° C. overnight and weighed to determine the theoretical content of the active agents.

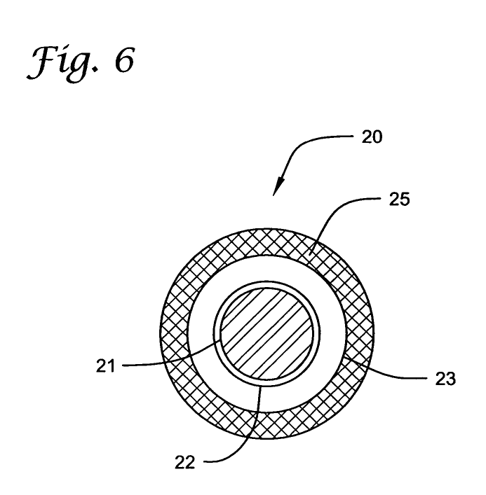

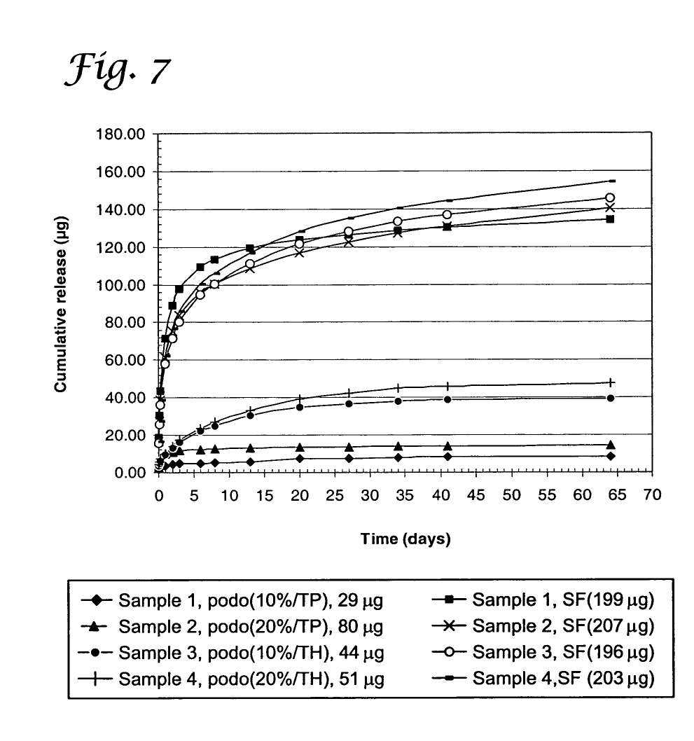

The design of this system 20 is shown in FIG. 6, wherein the stent wire 21 is coated with a primer layer 22, which is coated with an inner layer 23 of a TECOPLAST or TECOTHANE polyurethane with podophyllotoxin therein. Over the inner layer 23 is an outer layer 25 of a TECOPLAST/PEVA blend with sulfasalazine therein. The primer layer 12 can include, for example, about 25 micrograms (μg) to about 50 micrograms of the primer. The release characteristics of the active agents from this system are shown in FIG. 7. In FIG. 7, the samples were fabricated such that the inner layer for sample 1 had about 10% of podophyllotoxin in TECOPLAST polyurethane; sample 2 had about 20% of podophyllotoxin in TECOPLAST polyurethane; sample 3 had about 10% of podophyllotoxin in TECOTHANE 75D polyurethane and sample 4 had roughly 20% of podophyllotoxin in TECOTHANE 75D polyurethane. For all samples, the outer layer was a blend of TECOPLAST/PEVA with 30% sulfasalazine.

### Example 5

Primed stents were prepared as in Example 1. Next, each stent was sprayed with 1% solution of etoposide/sulfasalazine (Sigma-Aldrich) in TECOTHANE 75D polyurethane (20% etoposide (EP), 10% sulfasalazine (SF), and 70% polymer) using THF as solvent. This represents the inner layer with a target coating of 600 μg+/−10% and a thickness of approximately 6 μm. The stent was dried in nitrogen environment at ambient temperature for 24 hours then vacuum-dried at 23° C. overnight and then weighed.

Next, the stent was sprayed with a 1% solution of 20% sulfasalazine (Sigma-Aldrich) in blends of 40/60% (Blend 2), and 20/80% (Blend 1) of copolymers designated C10 and C19 in chloroform to form the outer layer. Polymer C10 is a copolymer containing 95% butyl methacrylate and 5% vinyl acetate. Polymer C19 is a copolymer containing 8% vinyl acetate, 74% hexyl methacrylate, and 18% n-vinyl pyrollidone.

Both C10 and C19 were prepared using methods known to those skilled in the art of polymer chemistry and as detailed in references such as, A. Ravve. Principles of Polymer Chemistry, Second Edition. 2000. Kluwer Academic/Plenum Publishers, New York. ISBN 0-306-46368-7; H. Allcock and F. Lampe. Contemporary Polymer Chemistry. 1981. Prentice-Hall, N.J. ISBN 0-13-170258-0. and A. Tonelli. Polymers from the Inside Out. 2001. Wiley-Interscience. ISBN 0-471-38138-1. Although methods used to synthesize the polymers of the present invention are known to those skilled in the art of polymer chemistry, additional details are provided herein for convenience.

In the first step of the synthesis, predetermined amounts of n-butyl methacrylate (BMA) and vinyl acetate (VAc) were mixed in a pre-dried glass reactor equipped for mechanical stirring while providing a nitrogen environment about the reactants. The mixture was then sparged with nitrogen for about five minutes. A requisite amount of azo-bis-butyronitrile (Azo) was added to the mixture. In most cases, isopropyl alcohol (IPA) sparged with nitrogen was also added to the mixture. The mixture was heated to the desired temperature under nitrogen and stirred for a certain period of time until the commencement of the second step.

In the second step of the synthesis, a second aliquot of the Azo free radical initiator and IPA were added prior to introduction of a second charge of monomer or comonomer. The monomer and comonomer were also sparged with nitrogen. The polymerization was continued at the desired temperature until monomer consumption practically ceased, maintaining agitation while possible.

At the conclusion of the second step, the heating was stopped and the product was mixed in the reactor with a suitable solvent such as acetone to facilitate the polymer purification by precipitation in a cold non-solvent such as water or methanol or a mixture thereof. The precipitated copolymer was then isolated by filtration and allowed to dry in a laminar flow hood under reduced pressure at room temperature until a constant dry weight was achieved. Further drying can be accomplished by heating under reduced pressure until a constant dry weight is achieved.

The target for the outer layer/coat was 600 μg+/−10% or roughly 6 μm in thickness. The stent was again dried in nitrogen environment at ambient temperature for 24 hours then vacuum-dried in an oven at 23° C. overnight and weighed to determine the theoretical content of the active agents. The amount of each active agent in each dual release system was determined by High Performance Liquid Chromotography(HPLC) using wavelength of 284 nanometer(nm) for etoposide and 362 nm for sulfasalazine.

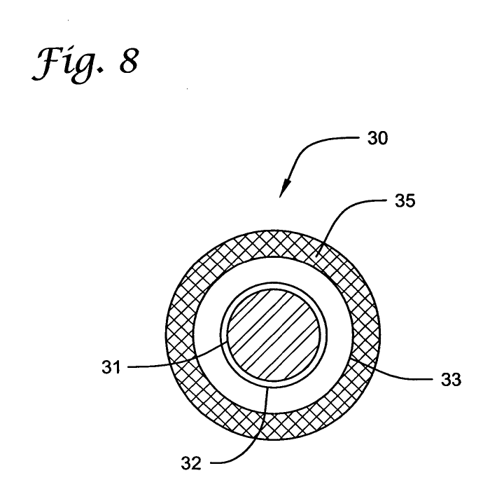

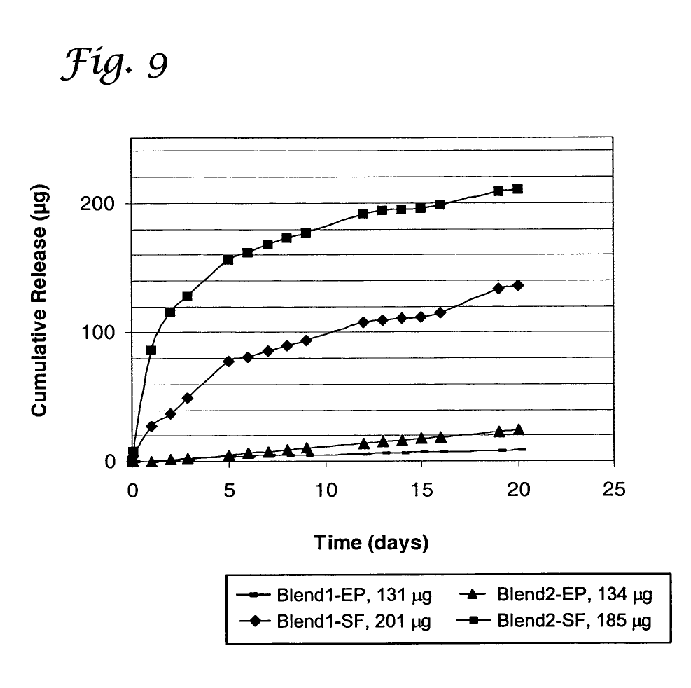

The design of this system 30 is shown in FIG. 8, wherein the stent wire 31 is coated with a primer layer 32, which is coated with an inner layer 33 of a TECOTHANE polyurethane with etoposide and sulfasalazine therein. Over the inner layer 33 is an outer layer 35 of a polymer blend between the two polymers C10 and C19 with sulfasalazine therein. The primer layer 32 can include, for example, about 25 micrograms (μg) to about 100 micrograms of the primer. The release characteristics of active agents from this system are shown in FIG. 9.

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

1. An active agent delivery system comprising:
two or more active agents and two or more layers of polymers;
wherein at least one layer comprises a miscible polymer blend comprising two or more miscible polymers; and
wherein delivery of at least one active agent occurs predominantly under permeation control.

2. The system of claim 1 wherein at least one active agent is incorporated within the at least one miscible polymer blend layer.

3. The system of claim 1 wherein the miscible polymer blend initially provides a barrier for the active agent.

4. The system of claim 3 wherein at least one active agent is incorporated within at least one inner layer.

5. The system of claim 3 wherein the barrier layer is an intermediate layer within a system comprising three or more layers.

6. The system of claim 3 wherein the barrier layer is the outermost layer of the system.

7. The system of claim 1 comprising at least 100 layers.

8. The system of claim 1 wherein the outermost layer comprises a single polymer.

9. The system of claim 1 wherein the miscible polymer blend forms an inner layer with at least one active agent incorporated therein.

10. The system of claim 9 wherein the inner layer comprising a miscible polymer blend with at least one active agent incorporated therein is the innermost layer.

11. The system of claim 10 further comprising a barrier layer overlying the miscible polymer blend layer, wherein the barrier layer does not include an active agent initially, and further wherein the barrier layer comprises a single polymer or a miscible polymer blend.

12. The system of claim 11 further comprising a layer overlying the barrier layer, wherein the overlying layer comprises at least one polymer and at least one active agent incorporated therein.

13. The system of claim 12 further comprising an outermost layer that does not initially include an active agent therein.

14. The system of claim 1 comprising at least two layers, wherein an inner layer comprises at least one polymer with at least one active agent incorporated therein, and the system further comprises a barrier layer overlying the inner polymer layer, wherein the barrier layer does not include an active agent initially.

15. The system of claim 14 wherein the barrier layer comprises a miscible polymer blend.

16. The system of claim 14 wherein the inner layer comprises a miscible polymer blend.

17. The system of claim 14 wherein the inner layer comprising a miscible polymer blend is the innermost layer.

18. The system of claim 14 further comprising at least two inner layers each of which comprise at least one polymer with at least one active agent incorporated therein.

19. The system of claim 14 further comprising an outermost layer that includes at least one polymer and at least one active agent incorporated therein.

20. The system of claim 1 comprising at least three layers.

21. The system of claim 20 wherein each of the layers comprises at least one active agent incorporated therein.

22. The system of claim 1 wherein each of the layers comprises at least one active agent incorporated therein.

23. The system of claim 1 wherein at least one of the layers does not initially include an active agent therein.

24. The system of claim 23 wherein the layer that does not initially include an active agent therein is the outermost layer.

25. The system of claim 23 wherein at least two layers do not initially include an active agent therein.

26. The system of claim 25 wherein the outermost layer does not initially include an active agent therein.

27. The system of claim 1 comprising at least two layers, wherein an inner layer comprises a single polymer with an active agent incorporated therein, and the system further comprises a barrier layer overlying the single polymer layer, wherein the barrier layer comprises the miscible polymer blend.

28. The system of claim 1 comprising at least two layers, wherein an inner layer comprises an immiscible mixture of two or more polymers with at least one active agent incorporated therein, and the system further comprises a barrier layer overlying the immiscible polymer mixture layer, wherein the barrier layer does not include an active agent initially, and further wherein the barrier layer comprises a miscible polymer blend.

29. The system of claim 28 wherein the inner layer comprising an immiscible mixture of two or more polymers with at least one active agent incorporated therein is the innermost layer.

30. The system of claim 28 further comprising a layer overlying the barrier layer, wherein the overlying layer comprises at least one polymer and at least one active agent incorporated therein.

31. The system of claim 1 wherein at least one active agent is incorporated within each layer except for the outermost layer.

32. The system of claim 1 wherein each layer comprises a blend of two or more miscible polymers.

33. The system of claim 32 wherein at least one active agent is incorporated within each layer.

34. The system of claim 1 comprising at least two layers, wherein an inner layer comprises a single polymer with at least one active agent incorporated therein, and the system further comprises an outermost barrier layer, wherein the barrier layer does not include an active agent initially, and further wherein the barrier layer comprises a miscible polymer blend.

35. The system of claim 34 wherein the inner layer comprising a single polymer and at least one active agent incorporated therein is the innermost layer.

36. The system of claim 1 comprising at least two layers, wherein an inner layer comprises an immiscible mixture of two or more polymers with at least one active agent incorporated therein, and the system further comprises an outermost barrier layer, wherein the barrier layer does not include an active agent initially, and further wherein the barrier layer comprises a miscible polymer blend.

37. The system of claim 36 wherein the inner layer comprising an immiscible mixture of two or more polymers and at least one active agent incorporated therein is the innermost layer.

38. The system of claim 1 comprising at least two layers, wherein an inner layer comprises a miscible blend of two or more polymers with at least one active agent incorporated therein, and the system further comprises an outermost barrier layer, wherein the barrier layer does not include an active agent initially, and further wherein the barrier layer comprises a miscible polymer blend.

39. The system of claim 38 wherein the inner layer comprising a miscible blend of two or more polymers and at least one active agent incorporated therein is the innermost layer.

40. The system of claim 1 wherein the concentration of at least one active agent varies throughout the layers to form a concentration gradient.

41. The system of claim 1 wherein the same polymers are used in each layer in varying amounts such that a concentration gradient is formed.

42. The active agent delivery system of claim 1 wherein the difference between the solubility parameter of the active agent that is to be released faster and to be present in a greater amount and the volume average solubility parameter of the blend of the two or more miscible polymers is smaller than the differences between the solubility parameter of each of the other one or more active agents and the volume average solubility parameter of the blend of the two or more miscible blends.

43. The system of claim 1 wherein a first active agent is selected from the group consisting of indomethacin, sulindac, diclofenal, etodolac, meclofenate, mefenamic acid, nambunetone, piroxicam, phenylgutazone, meloxicam, dexamethoasone, betamethasone, dipropionate, diflorsasone diacetate, clobetasol propionate, galobetasol propionate, amcinomide, beclomethasone dipropionate, fluocinomide, betamethasone valerate, triamcinolone acetonide, penicillamine, hydroxychloroquine, sulfasalazine, azathioprine, minocycline, cyclophosphamide, methotrexate, cyclosporine, leflunomide, etanercept, infliximab, ascomycin, beta-estradiol, rosiglitazone, troglitazone, pioglitazone, S-nitrosoglutathione, gliotoxin G, panepoxydone, cycloepoxydon tepoxalin, curcumin, a proteasome inhibitor, antisense c-myc, celocoxib, valdecoxib, and combinations thereof.

44. The system of claim 43 wherein a second active agent is released at a slower rate than that of the first active agent, after the start of release of the first active agent, or both.

45. The system of claim 44 wherein the second active agent is selected from the group consisting of podophyllotoxin, mycophenolic acid, teniposide, etoposide, trans-retinoic acids, 9-cis retinoic acid, 13-cis retinoic acid, rapamycin, a rapalog, camptothecin, irinotecan, topotecan, tacromilus, mithramycin, mitobronitol, thiotepa, treosulfan, estramusting, chlormethine, carmustine, lomustine, busultan, mephalan, chlorambucil, ifosfamide, cyclophosphamide, doxorubicin, epirubicin, aclarubicin, daunorubicin, mitosanthrone, bleomycin, cepecitabine, cytarabine, fludarabine, cladribine, gemtabine, 5-fluorouracil, mercaptopurine, tioguanine, vinblastine, vincristine, vindesine, vinorelbine, amsacrine, bexarotene, crisantaspase, decarbasine, hydrosycarbamide, pentostatin, carboplatin, cisplatin, oxiplatin, procarbazine, paclitaxel, docetaxel, epothilone A, epothilone B, epothilone D, baxiliximab, daclizumab, interferon alpha, interferon beta, maytansine, and combinations thereof.

46. The system of claim 1 wherein at least one active agent is selected from the group consisting of podophyllotoxin, mycophenolic acid, teniposide, etoposide, camptothecin, irinotecan, topotecan, mithramycin, and combinations thereof.

47. The system of claim 46 further wherein one active agent is sulfasalzine.

48. The system of claim 46 further wherein one active agent is indomethacin.

49. The system of claim 46 further wherein one active agent is ascomycin.

50. The system of claim 46 further wherein one active agent is leflunomide.

51. The system of claim 46 further wherein one active agent is dexamethasone.

52. The system of claim 46 further wherein one active agent is piroxicam.

53. The system of claim 46 further wherein one active agent is beclomethasone dipropionate.

54. The system of claim 46 further wherein one active agent is S-nitrosoglutathione.

55. The system of claim 1 wherein at least one active agent is selected from the group consisting of trans-retinoic acids, 9-cis retinoic acid, 13-cis retinoic acid, etoposide, mycophenolic acid, podophyllotoxin, teniposide, camptothecin, irinotecan, topotecan, mithranycin, and combinations thereof.

56. The system of claim 55 further wherein one active agent is rosiglitazone.

57. The system of claim 55 further wherein one active agent is troglitazone.

58. The system of claim 55 further wherein one active agent is pioglitazone.

59. An active agent delivery system comprising:
two or more active agents;
two or more layers of polymers; and
an optional barrier layer;
wherein at least one layer comprises a miscible polymer blend comprising two or more miscible polymers with at least one active agent incorporated therein; and
wherein delivery of at least one active agent occurs predominantly under permeation control.

60. The system of claim 59 wherein the miscible polymer blend layer with the at least one active agent incorporated therein is an inner layer.

61. The system of claim 59 wherein the barrier layer is an intermediate layer within a system comprising three or more layers.

62. The system of claim 59 wherein the barrier layer is the outermost layer of the system.

63. A medical device comprising the active agent delivery system of claim 1.

64. The medical device of claim 63 selected from the group consisting of a stent, stent graft, anastomotic connector, lead, needle, guide wire, catheter, sensor, surgical instrument, angioplasty balloon, wound drain, shunt, tubing, urethral insert, pellet, implant, blood oxygenator, pump, vascular graft, valve, pacemaker, orthopedic device, replacement device for nucleus pulposus, and intraocular lense.

65. A medical device comprising the active agent delivery system of claim 59.

66. The medical device of claim 65 selected from the group consisting of a stent, stent graft, anastomotic connector, lead, needle, guide wire, catheter, sensor, surgical instrument, angioplasty balloon, wound drain, shunt, tubing, urethral insert, pellet, implant, blood oxygenator, pump, vascular graft, valve, pacemaker, orthopedic device, replacement device for nucleus pulposus, and intraocular lense.

67. A stent comprising the active agent delivery system of claim 1.

68. A stent comprising the active agent delivery system of claim 59.

69. A medical device comprising:
a substrate surface;
a polymeric undercoat layer adhered to the substrate surface; and
an active agent delivery system adhered to the polymeric undercoat layer;
wherein the active agent delivery system comprises:
two or more active agents and two or more layers of polymers;
wherein at least one layer comprises a miscible polymer blend comprising two or more miscible polymers; and
wherein delivery of at least one active agent occurs predominantly under permeation control.

70. A stent comprising:
a substrate surface;
a polymeric undercoat layer adhered to the substrate surface; and
an active agent delivery system adhered to the polymeric undercoat layer;
wherein the active agent delivery system comprises:
two or more active agents and two or more layers of polymers;
wherein at least one layer comprises a miscible polymer blend comprising two or more miscible polymers; and
wherein delivery of at least one active agent occurs predominantly under permeation control.

71. A method for delivering two or more active agents to a subject, the method comprising:
providing an active agent delivery system of claim 1; and
contacting the active agent delivery system with a bodily fluid, organ, or tissue of a subject.
